(19) United States
(12) Patent Application Publication
(10) Pub. No.: US 2005/0014153 A1
(43) Pub. Date: Jan. 20, 2005

(54) Reaction chambers containing complex stable reagent formulations and test kit for detection and isolation of pathogenic microbial nucleic acids

(76) Inventors: Timo HIillebrand, Honow (DE); Thomas Kohler, Borsdorf (DE); Peter Bendzko, Berlin (DE)

Correspondence Address: Norris McLaughin & Marcus, 30th Floor, 220 East 42nd Street, New York, NY, 10017, US

(21) Appl. No.: 10/493,287

(22) PCT Filed: Nov. 4, 2002
(86) PCT No.: PCT/DE02/04081

(30) Foreign Application Priority Data
Nov. 2, 2001 (DE) 201 17 746.3

(51) Int. Cl. C12Q 1/68; C12M 1/34

(52) U.S. Cl. 435/6; 435/287.2

(57) ABSTRACT

The invention relates to standard reaction areas containing complex, storage-stable reagent formulations and thus suited to acceptance and, if need be, storage of complex liquid patient samples, thereafter to be examined for the existence of pathogenic nucleic acids (bacteria, viruses etc.). Preferably, the reaction areas manifest complex, storage-stable reagent formulations permitting detection and quantitative isolation of viral and bacterial nucleic acids in a test kit, even in the existence of extremely low copy numbers from a complex biological sample. At the same time, a simple archiving system for the clinically relevant nucleic acids can be provided. Thanks to the use of said reaction areas containing the complex, storage-stable reagent formulations, necessary process steps of sample handling are drastically reduced and thus potential risks of infection and risks of contamination distinctly lowered.

The invention relates to standard reaction areas containing complex, storage-stable reagent formulations and thus suited to accept and, if need be, store complex fluid patient samples, thereafter to be examined for the existence of pathogenic nucleic acids (bacteria, viruses etc.). Preferably, the reaction areas manifest complex, storage-stable reagent formulations permitting detection and quantitative isolation of viral and bacterial nucleic acids in a test kit, even in the existence of extremely low copy numbers from a complex biological sample. At the same time, a simple archiving system for the clinically relevant nucleic acids can be provided. Thanks to the use of said reaction areas containing the complex, storage-stable reagent formulations, necessary process steps of sample handling are drastically reduced and thus potential risks of infection and risks of contamination distinctly lowered.

Screening of complex biological samples (serum, plasma, blood etc.) for the existence of infectious components is gaining more and more importance. Virus infections such as HIV, HCV or HBV are becoming more and more widespread all over the world, New test methods on the basis of the use of sensitive amplification techniques such as PCR or NASBA enable a highly efficient detection of viruses and are being used more and more frequently as diagnostic instruments. Specialists know that an essential step for the application of these techniques for the detection of pathogenic nucleic acids in the isolation of the nucleic acids (RNA or DNA) comprises relevant complex clinical samples. Without such a highly efficient isolation, e.g. of viral nucleic acids, no sufficiently sensitive diagnosis can be carried out.

At the present, the isolation, e.g. of viral nucleic acids from blood products, is customarily done via the lysis of the original material with a buffer containing chaotropic components of a high ion strength and the subsequent binding of the nucleic acids to a solid phase (e.g. membrane filter). The bound nucleic acids are washed on the solid phase and finally dissolved from the solid phase with a buffer of a suitable ion strength.

Inter alia, the process is portrayed in patent U.S. Pat. No. 5,234,809 A and known world-wide under the name of “Boom Patent”. The process precisely describes the isolation of nucleic acids from original materials containing the latter by incubation of the original material with a chaotropic buffer and a solid phase binding DNA. The chaotropic buffers implement both the lysis of the original materials and also the binding of the nucleic acids to the solid phase. The method is well suited to isolating nucleic acids from small sample quantities and is applied in practice specifically in the area of the isolation of viral nucleic acids.

One main problem in the isolation of viral and bacterial nucleic acids is the realisation of a sufficiently high diagnostic sensitivity, as the number of viral copies (or the copy number of other microbial pathogens) in a complex biological sample is very low as a rule. Solutions to increase the extraction efficiency up to now relate to the increase of the volume of the clinical sample to be examined or the enrichment of viral particles by centrifugation techniques. It becomes clear to specialists that narrow borders are set for these variants.

An increase of the initial volume of the clinical sample with the extraction methods currently in use leads to a necessary proportional or also over-proportional increase of the necessary extraction buffers. This subsequently results in a multiplication of necessary centrifugation steps in order to transfer the increased volume of the sample to be processed to a centrifugation filter on which the binding of the nucleic acid to be bound is to take place in the end.

In this way, the manual efforts for the extraction, the extraction time and the contamination risk are increased.

A further problem in the isolation, in particular of viral nucleic acids, for a subsequent diagnostic detection is that a series of reaction components has to be pipetted into a reaction vessel containing the sample for the extraction of the nucleic acids.

The task of the invention in question was thus to look for possibilities removing all the existing problems in connection with the handling of a complex biological sample to be investigated for the existence of microbial nucleic acids and to find reaction approaches permitting a simplification of the methods for the isolation of microbial nucleic acids from patients' samples and further automating the methods, in order to enable a parallel processing of samples at high throughflow. Further, known risks of cross-contaminations are to be distinctly reduced with these reaction approaches via the reduction of working steps.

The invention is realised by claims 1, 8, 11 and 12, the sub-claims being preferential variants.

The task was solved by standard reaction vessels (such as 1.5 ml or 2.0 ml Eppendorf reaction vessels or 96-well or 386-well micro titre plates) containing all the components suited to accept and, if need be, store complex fluid patient samples, with the samples subsequently to be examined (at any point in time) for the existence of pathogenic microbial nucleic acids (bacteria, viruses etc.). I.e., reaction areas are provided which are needed for the lysis of a biological sample for the extraction methods used according to the state of the art and the diagnostic detection of microbial nucleic acids. Preferably, these are reaction areas manifesting all the components necessary for the isolation of pathogenic nucleic acids from patient samples in a complex, storage-stable formulation:

In particular, this is a question, for the detection of microbial nucleic acids, of:

standard reaction areas entailing

1. at least one standard nucleic acid (control standard to verify the extraction efficiency)
2. at least one carrier nucleic acid (necessary in order to be able to extract even the slightest quantities of microbial nucleic acid)
3. a lysis buffer formulation and
4. if need be, a proteolytic enzyme (e.g. a proteinase).

All these components are contained in the reaction cavities as mainly or completely water-free reagent formulations and manifest practically no volume due to their slight quantity. The production of the complex, storage-stable reagent formulations is done by specialists, preferably either by vacuum drying or by lyophilisation.

The reaction areas prepared in this way have numerous benefits. In particular, they are suited as integral parts of test kits for detection and isolation of pathogenic nucleic acids. For example, they enable the processing of a complex biological sample with the objective of the quantitative detection of pathogenic nucleic acids, which starts by the transfer of the sample into the reaction vessel according to the invention. All the components for the lysis of the original material and extraction standards necessary for a quantitative nucleic acid diagnosis are already in the reaction vessel. In this way, no further pipetting steps for the addition of buffers or other essential components are necessary any more. Thus, the necessary “hands-on” efforts are drastically reduced with a simultaneous distinct reduction of the contamination risk. The more samples are to be processed parallel, the more important this becomes. It also becomes clear to a specialist that automation is made very simple by the inclusion of the reaction materials according to the invention.

A further essential benefit is the fact that the volume of the clinical sample to be examined can be distinctly increased by using the reaction areas according to the invention. Thanks to the complete lack of liquid reaction components, the volume of the sample added finally also corresponds to the volume of the overall reaction. The multiple loading steps of centrifugation filters necessary up to now in an increase of the volume of the biological sample are no longer necessary. After the lysis of the original material, the lysate is mixed with defined shares, e.g. of an alcohol, and subsequently passed over a solid phase which is in a position to bind the nucleic acid of the sample, The solid phase is then washed with washing buffers containing ethanol and the nucleic acid of the sample, including the extraction standard, released from the solid phase by addition of a low-salt buffer. The nucleic acid is now available for a subsequent quantitative analysis.

The test kit according to the invention entails not only the reaction areas, but also
a solid phase known to specialists for the binding of nucleic acids,
if need be, binding buffers known per se,
if need be, washing buffers known per se,
if need be, low-salt buffers known per se and also, if need be, further ancillaries also known to a specialist.

Surprisingly, it is also seen that the nucleic acid from the samples remains stable on the solid phase for a long time after the necessary washing steps and a subsequent drying of the solid phase. This has the advantage that the nucleic acid in this bound form can be stored or even dispatched at ambient temperature without any problems. Complicated long-term storages of nucleic acid from samples at −80° C. and also storage with addition of ethanol are thus no longer necessary. If required, the nucleic acid from the samples is simply removed from the solid phase by addition of a low-salt buffer and transferred to diagnostics.

Surprisingly, RNA can also be stored for such long periods without the occurrence of degradations and can be removed from the solid phase at any required point in time. In this way, a completely new archiving system for nucleic acid from samples is available.

The solid phases are customarily integral parts of filtration units, which can be available as “single tube” and also as multiple variants of individual reaction cavities (e.g. 96-well filtration plates, 384-well filtration plates etc.). They are thus compatible with the reaction cavities according to the invention and permit the isolation or archiving of nucleic acid from samples in varying formats.

A further advantage of the reaction areas used is seen in the fact that the nucleic acid contained is protected under the used lysis buffer formulations and added carrier nucleic acids following addition of the fluid patient sample. This permits transport of the sample without cooling and thus also considerably facilitates the logistics of sample collection and sample dispatch for a subsequent quantitative diagnosis, specifically of microbial nucleic acids. Finally, it is stated that the agent according to the invention can also be used for many other questions of molecular diagnosis and is thus not only restricted to use in microbial diagnosis.

The application of the reaction areas according to the invention is to be explained below in more detail with an example. Example of embodiment

### ISOLATION OF VIRAL NUCLEIC ACIDS FROM BODY FLUIDS (SERUM, PLASMA, URINE ETC.)

Mixing of 200 μl serum with 200 μl H₂O. Transfer of the sample to the reaction area according to the invention (2 ml reaction vessel) containing a storage-stable reagent formulation in a water-free or predominantly water-free form, comprising:
1. DNA and RNA of a synthetic gene (pMS 1; Springer Lab Manual; Quantification of mRNA by Polymerase Chain Reaction; Springer Verlag 1995) as a standard nucleic acid for the extraction control
2. tRNA as a carrier nucleic acid
3. Lysis buffer containing CTAB, polyvinylpyrrolidone, ammonium chloride, Tris-HCl, proteinase K

Mixing of the solution and incubation for 10 min at 56° C. in a thermo-mixer. Subsequent addition of 400 μl isopropyl alcohol and transfer of the solution to a centifugation column (e.g. centrifugation column of the firm of Invitek with fibre glass material contained). Centrifugation for 1 min and rejection of the filtrate. Double washing of the centrifugation column with washing buffers containing ethanol. Drying of the centrifugation column by 3-minute centrifugation.

Addition of 80 μl of an elution buffers (10 mM Tris HCl; pH 8.5) to the centrifugation column and centrifugation for 1 min.

Detection of the isolated viral RNA and/or DNA is done by means of known amplification techniques.

1. A reaction vessel for acceptance and storage of complex biological samples comprising complex, storage-stable reagent formulations in a solid form suitable for the isolating of nucleic acids.

2. The reaction_vessel_according to claim 1, further comprising standard reaction areas entailing
at least one standard nucleic acid
at least one carrier nucleic acid
a lysis buffer formulation;
wherein all components are present in a dry or substantially water-free state.

3. The reaction vessel according to claim 1 wherein the standard reaction areas are 1.5 ml or 2 ml reaction vessels, 96-well micro-titre plates or 384-well micro titre plates, with the individual wells containing identical or differing reagents.

4. The reaction_vessel_according to claim 1 comprising nucleic acids as control standards for the verification of the extraction efficiency.

5. The reaction_vessel_according to claim 1, comprising RNA, poly-RNA or DNA as carrier nucleic acids (for extracting small amounts of microbial nucleic acid).

6. The reaction_vessel_according to claim 1 further comprising detergents, mono or polyvalent cations, and at least one buffering component.

7. The reaction_vessel_according to claim 1, wherein they further comprise one or more proteolytic enzymes.

8-10. (Cancel).

11. Test kit for detection and quantitative isolation of microbial, preferably viral and bacterial nucleic acids, comprising,
reaction vessel according to one of the claims 1 to 7
a solid phase for the binding of the nucleic acids,
and optionally one or more components selected from the group consisting of
binding buffers
washing buffers and
low-salt buffers.

12. An archiving system for clinically relevant nucleic acids entailing a solid phase according to claim 11 manifesting the bound nucleic acid in a storage-stable form in a predominantly or completely dry state.

13. The reaction vessel of claim 7, wherein the proteolytic enzymes comprise proteinase K.

14. A method for using the reaction vessel of claim 1 for isolating or detecting nucleic acids comprising:
(a) providing the reaction vessel of claim 1, wherein the vessel comprises a dry nucleic acid-isolating formulation having a lysis buffer component;
(b) adding a biological sample comprising nucleic acids to the reaction vessel and an amount of water effective to solubilize the dry nucleic acid-isolating formulation; and
(c) incubating the biological sample under conditions effective for lysing the biological sample and release of the biological sample's nucleic acid molecules.

15. The method of claim 14 wherein the dry nucleic acid-isolating formulation comprises a known amount of control nucleic acid for monitoring the efficiency of the isolation

16. The method of claim 14 wherein the dry nucleic acid-isolating formulation comprises at least one proteolytic enzyme.

17. The method of claim 14, wherein the biological sample's nucleic acids are viral or bacterial.

18. The method of claim 14, wherein at least one of the biological sample's nucleic acids are selected from the group consisting of pathogenic organisms, yeast and protozoa.

19. The method of claim 14, wherein the biological sample's nucleic acids are microbial.
